(19) United States
(12) Patent Application Publication
(10) Pub. No.: US 2011/0276137 A1
(43) Pub. Date: Nov. 10, 2011

(54) TUBULAR IMPLANTABLE CORD

(76) Inventors: Bahaa Seedhom, Harrogate (GB); David Beevers, Harrogate (GB)

(21) Appl. No.: 13/126,676

(22) PCT Filed: Oct. 29, 2009
(86) PCT No.: PCT/GB2009/051459

§ 371 Date: Jul. 19, 2011

(30) Foreign Application Priority Data
Oct. 30, 2008 (GB) 0819912.7

(51) Int. Cl. A61F 2/08

(52) U.S. Cl. 623/13.11

(57) ABSTRACT

A surgical cord (100, 200,101)comprising a tubular section along its main length and comprising regions (600, 608, 601) exhibiting different mechanical and/or physical properties resultant from a change in material, pattern type, pattern density, yarn thickness and/or shape. Different regions along the length of the cord are optimised to provide the required extensibility, abrasion resistance and/or stiffness. A tissue core (904) may be incorporated within the tubular cord structure and a means of indicating the tensile loading force applied to the cord may be provided.

The present invention relates to an implantable surgical cord formed from overlapping yarn strands.

Because connective tissues are mostly avascular they have a poor capacity for healing and so their injuries are often repairable only via surgical procedures in which implantable connecting devices are used to reconnect or reattach torn tissue to its original anchor site.

An important consideration with connective devices such as sutures and tapes is the strength of repair. The repair strength is governed by the strength of the device and also its resistance to being pulled through the tissue when under load, whereby the suture might slip or cut through the tissue. Sutures have small diameters and flat tapes sharp edges which lead to such devices pulling, or cutting, through tissue. A surgical implantable connective device which resists tissue pull through would have improved function for numerous surgical applications.

The present invention provides a surgical cord in which the yarn strands are arranged to provide strength and resistance to stretch in response to the application of a tensile loading force. The surgical cord is also configured so as not to present sharp edges that would otherwise lead to tissue pull through when the implant is loaded post surgical implantation, while providing end regions that are easy to thread through eyelets of needles and easy for a surgeon to grasp, manipulate and apply tension so as to aid surgical implantation.

According to a first aspect of the present invention there is provided a surgical cord comprising: a plurality of overlapping yarn strands forming the walls of a substantially tubular region extending over a length of the cord.

The tubular region of the device may comprise a substantially circular, elliptical, or oval hollow cross sectional profile whilst the bordering substantially flat regions comprise a reduced cross sectional profile relative to the tubular region and are not hollow.

The structure of the device may be braided, woven or knitted, and formed from bundles of fibres that may be twisted together extend parallel and/or are crimped together. Preferably the structure is woven. However, it may comprise a combination of the aforementioned structures.

The yarn strands may be constructed from individual filaments. Each filament may be individually twisted, but preferably they are parallel and not twisted. These filaments may be braided, woven or twisted together to form the yarn. Preferably the filaments are twisted together. Additionally, the filaments may be crimped. Where the surgical cord comprises a woven pattern having warps, aligned substantially parallel to the longitudinal axis of the cord and wefts aligned transverse to the longitudinal axis, the warps and wefts may comprise the same or different yarn configurations comprising woven, braided, twisted and/or crimped filaments.

Discrete regions or sections along the length of the tube may comprise yarns made from different materials having different physical and/or mechanical properties. These regions of different materials may be formed as annular bands to give the cord different mechanical and/or physical properties along its length. A first region along the length may exhibit greater extensibility than a second region whilst the second region may exhibit greater stiffness and abrasion resistance. Alternatively or in addition, the different physical and/or mechanical properties of the cord at these discrete regions along its length may be provided by changing the relative thickness of the yarn strands, the overlap configuration including variation of the overlap pattern (e.g. braiding, intertwining, plaiting, linking, knotting, weaving, plying or twisting), porosity, density of the mesh/braid and the thickness and/or cross sectional profile of the yarn strands, a coating, manufacturing process that affects the bulk material (for example heat treatment) or surface treatment (for example gas plasma treatment). In particular, where the cord is woven, the number of warps and/or wefts at each of these specific regions may be different.

When the surgical cord is utilized, for example, as a prosthetic ligament, it could pass through regions of soft tissue such as tendon and regions of hard tissue such as bone. It is advantageous for the cord to exhibit different mechanical and/or physical properties along its length to correspond with such variations in the tissue through which the cord could pass. In particular, the filaments which lie in the direction of the longitudinal axis of the device at the end portions of the device which would locate adjacent to bone may be composed from a first material that exhibits abrasion resistance and a high resistance to extension. While the central section that is adjacent to tendon is composed from a second material that is more extensible than the first material. This would provide a device with relative inextensible extremities such that when the device is loaded the motion between the device and bone is reduced which would aid bone ingrowth into the device and hence provide enhanced fixation to the hard bone. Their abrasion resistance would further provide protection against potential sharp edges of bone which can lead to cutting of and rupture of the cord. The central tubular section would have properties to match the soft tissue that is being reconstructed.

The structure may be constructed from a variety of biocompatible materials including but not confined to surgical gut, silk, cotton, polyamide (Nylon), polyethylene terepthelate (polyester), polyethylene (PE), ultrahigh molecular weight polyethylene (UHMWPE), polypropylene (PP), polydioxanone, polyetheretherketone (PEEK), polytetrafluoroethylene (PTFE), polyaramid, or bioabsorbable materials. The device may be composed of a combination of the aforementioned materials. Preferably the device is constructed wholly from polyester.

In one alternative embodiment of the invention, where the cord is woven, the first material forming the warp yarns is a material such as UHMWPE or polyester and the second material forming the wefts is a bioabsorbable such as polyglycolic acid (PGA), polylactic acid (PLA) or co-polymer of such materials. This provides a device which retains its strength over time since all the load transmitting fibres are permanently implanted. This device has the advantage that it would gradually reduce in bulk as the non load bearing fibres resorb over time. The resorption of the non load bearing fibres has the advantage that further space is created for unrestricted tissue ingrowth. Moreover, fibres of differing resorption rates could be utilized so that the amount of space made available to the encroaching tissue ingrowth can be governed over time.

Such a structure could be manufactured by forming yarns which are composed of interlinking loops of different materials. Many configurations of interlinking loops can be used. Materials may also be linked by air entanglement or air splicing. The distinction between adjacent sectors of materials may be abrupt, or a gradual change may be incorporated.

The cord may comprise multiple regions along its length in which the yarn strands comprise the same or different material. In particular, regions or bands of the cord that are intended to be secured to bone may comprise a material that is inextensible, inflexible and exhibits abrasion resistance. Such materials include stainless steel, titanium or synthetic polymers optionally with or without coatings to enhance stiffness. Conversely, the sections of the cord along its length that are configured to simulate biological tissue at a joint region may be made from a flexible extensible material. This type of configuration involving multiple changes of material along the length of the cord may be provided by interlinked loops of yarn strands formed from twisted filaments.

In one alternative embodiment of the invention, the first material is a material such as UHMWPE or polyester and the second material is a bioabsorbable which incorporates a drug, a growth factor, or other biologically active substance. This material may be formed by impregnating the substance within the bioabsorbable material. Alternatively, the substance may be coated on the bioabsorbable material. Alternatively, the bioabsorbable material may be coated in the substance. Alternatively the substance and the polymer may be combined prior to or during fibre spinning. The change in material may be within the filaments/yarn strands which lie in the perpendicular direction of the longitudinal axis of the device (wefts where the cord is a woven mesh structure). This provides a device which retains its strength over time since all the load transmitting fibres are permanently implanted. This device has the advantage that the non-load bearing filaments perpendicular to the longitudinal axis would gradually resorb and release a substance. Moreover, fibres of differing resorption rates could be utilized to provide a timed substance release delivery system. By way of example, the filaments in the second material may comprise three fibres, each of different absorption rates. This would provide a system whereby the first would be a quickly absorbing fibre that would release a substance soon after implantation, the second fibre would resorb three months after implantation to provide a further or different dose of a substance, and the third fibre would resorb six months after implantation to provide another or different dose.

In one alternative embodiment of the invention the material in the sector of the device which lies adjacent to the bone tunnels would be one which is permanent or bioabsorbable which incorporates a drug, growth factor, surface treatment or a substance which is oseoinductive or osteoconductive, so promoting bone in growth into the device to provide fixation. Materials typically include tricalcium phosphate, hydroxyapatite or poly lactide carbonate. The material in the sector of the device which lies between bone anchor sites may be one that incorporates a drug, growth factor, surface treatment or a substance to enhance cell adherence and proliferation to encourage tissue ingrowth to reconstruct the soft tissue, for example a ligament or tendon.

Alternatively, or in addition to incorporating different materials, the amount of material may change along the cord length to affect its physical and/or mechanical properties. This may increase or reduce the bulk or diameter of the material at various sections of the cord. This increase in material may provide protection from external objects or edges, changes in properties such as strength or extension, or provide a means of fixation by using a stepped bone tunnel so that the increased diameter section of the device wedges against the smaller section. The increase in diameter may be provided by using yarns created by linking loops together, where one of the loops contains more material than adjacent loops. Materials may also be added by air entanglement or air splicing.

Optionally, the yarn strands and in particular at least one of the filaments/yarn strands that lie in the perpendicular direction of the longitudinal axis of the device is made from a material that is radio opaque. This filament is configured to form an identifiable trace on an X-ray. Since the filament would spiral through the wall thickness, when assembled as a woven structure or braid for example, it would identify the entire outer diameter or bulk of the ligament and hence the surgeon can determine on a post operative X-ray whether the device has been implanted in the correct position.

Preferably, the cord comprises one or more fibres of contrasting colors. This may be provided by dying fibres of polyester or polyamide (Nylon), or a monofilament of colour extruded polypropylene or polydioxanone. These high visibility fibres would be used to identify the transitions between different materials or changes in structural properties in a device with a composite structure. These coloured fibres could also mark the apertures, so enabling the surgeon to easily identify the holes in the device through which the tissue can be threaded and consequently ease the surgical procedure. Without such coloured marking fibres identification between the adjacent sectors of UHMWPE fibres and PET fibres would be difficult as both materials are typically white or translucent. Each feature could be identified by a fibre of a different colour; by way of example a blue fibre could be used to identify the apertures, while a black fibre used to identify material changes.

Optionally, at least one of the fibres that lie in the direction of the longitudinal axis of the device is coloured. This coloured fibre forms an identifiable trace along the length of the device to enhance visibility during surgery.

According to a second aspect of the present invention there is provided a woven surgical cord comprising: a plurality of yarn strands having warps aligned with the longitudinal axis of said cord that overlap wefts aligned transverse to the longitudinal axis of said cord; said yarn strands at a first region extending over a portion of the length of the cord comprising a first material and; said yarn strands at a second region extending over a portion of the length of the cord comprising a second material.

According a third aspect of the present invention there is provided a braided surgical cord comprising: a plurality of yarn strands overlapping to form a braided pattern; said yarn strands at a first region extending over a portion of the length of the cord comprising a first material and; said yarn strands at a second region extending over a portion of the length of the cord comprising a second material.

According to a fourth aspect of the present invention there is provided a knitted surgical cord comprising: a plurality of yarn strands overlapping to form a knitted pattern; said yarn strands at a first region extending over a portion of the length of the cord comprising a first material and; said yarn strands at a second region extending over a portion of the length of the cord comprising a second material.

Optionally, the cord being either a woven, knitted or braided structure may comprise regions along its length having a different overlapping weave pattern selected from any one or a combination of a woven, knitted or braided structure. The different overlapping pattern may be formed integrally with the main length of the cord or may be supplementary to the overlapping yarn strands that extend over the main length of the cord. That is, the additional woven, knitted or braided pattern may be overlaid on top of the yarn strands upon which the cord is formed.

Further details of the present invention will now be described by way of example only with reference to the accompanying drawings in which:

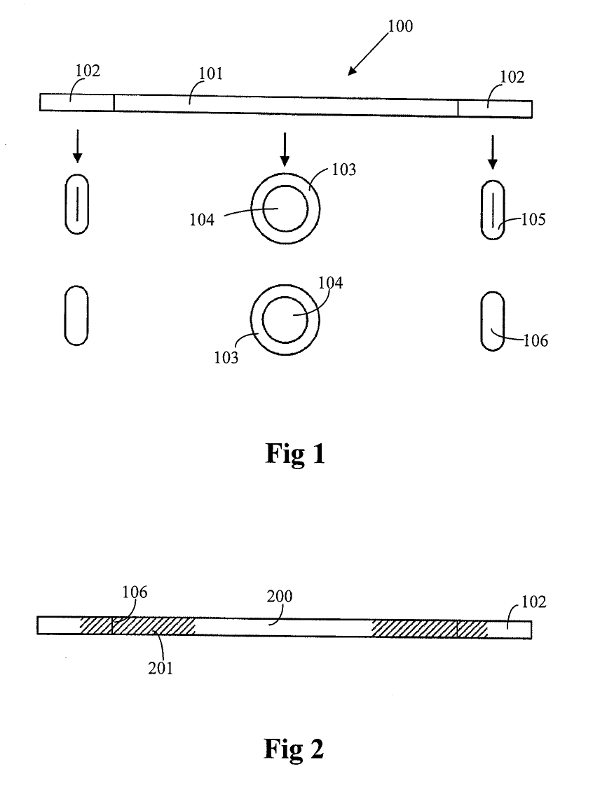

FIG. 1 is a schematic illustration of the surgical cord according to a specific implementation of the present invention;

FIG. 2 is a schematic illustration of the medical cord comprising different material segments along its length according to a specific implementation;

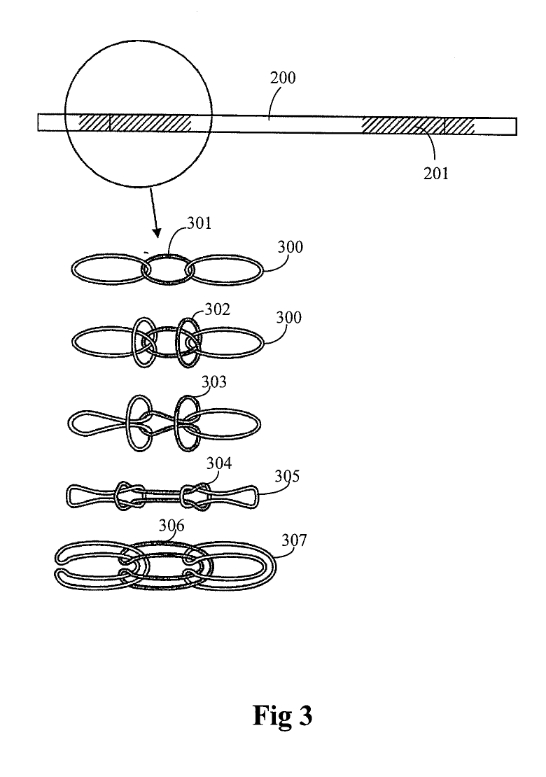

FIG. 3 illustrates the medical cord of FIG. 2 and the various loop constructions that may form the mesh structure;

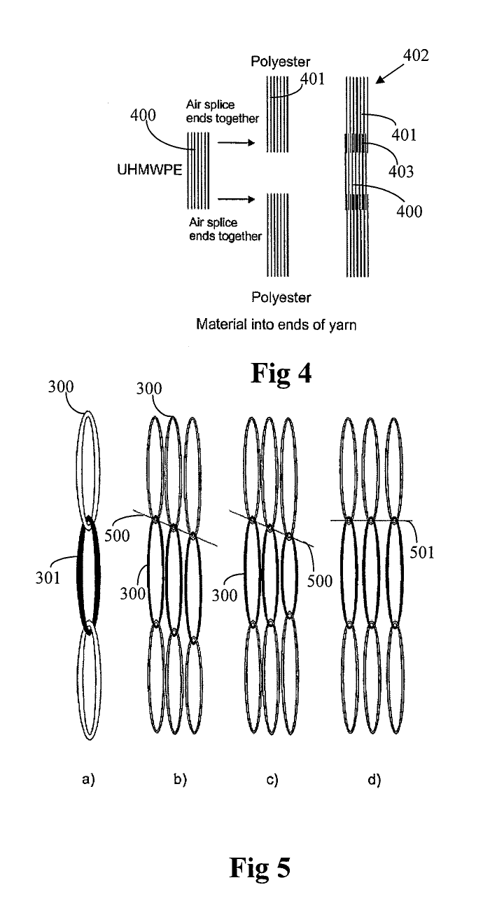

FIG. 4 illustrates schematically an air splice process for constructing the medical tape;

FIG. 5 illustrates variations on the looped interface of the different materials of the medical cord;

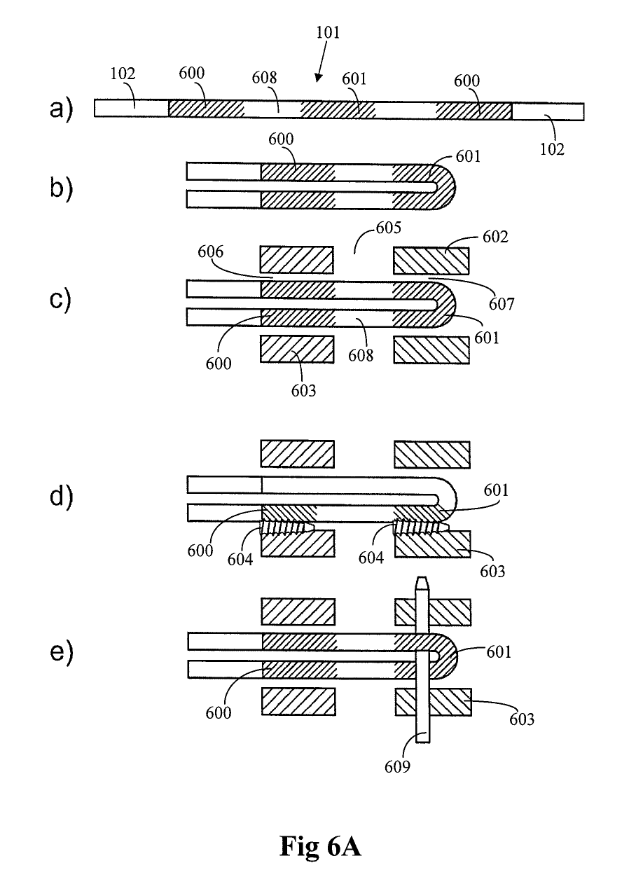

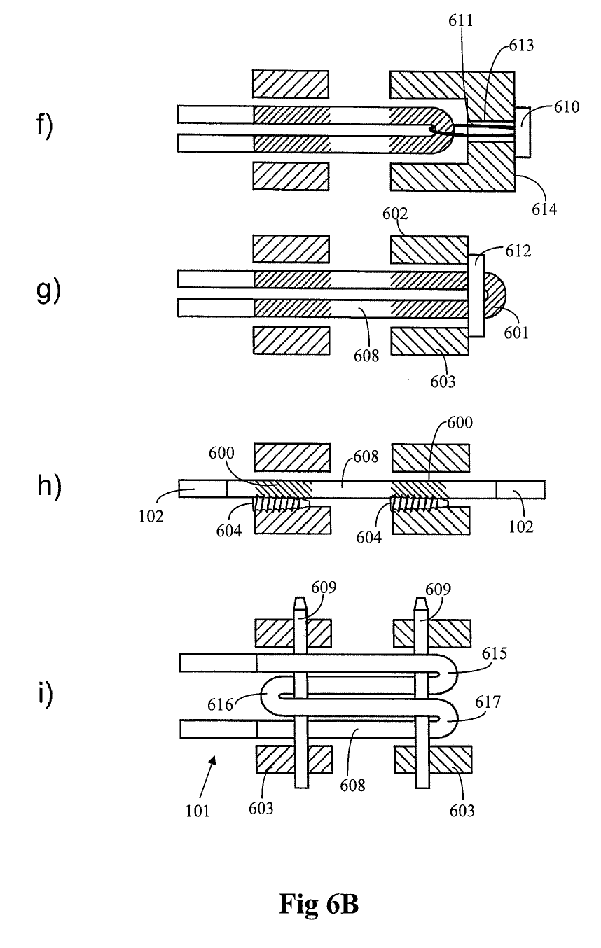

FIG. 6 illustrates schematically the medical cord comprising different material sections along its length and relative positioning at a plurality of anchor sites;

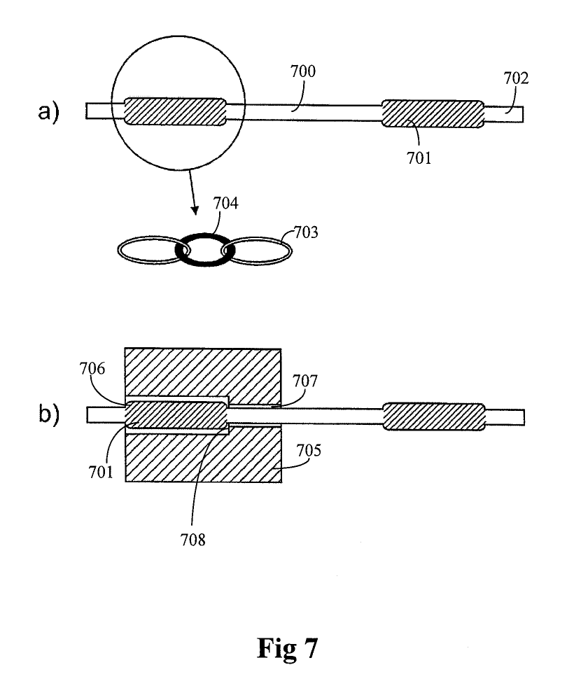

FIG. 7 illustrates the medical cord comprising a different cross sectional profile at different region segments along its length;

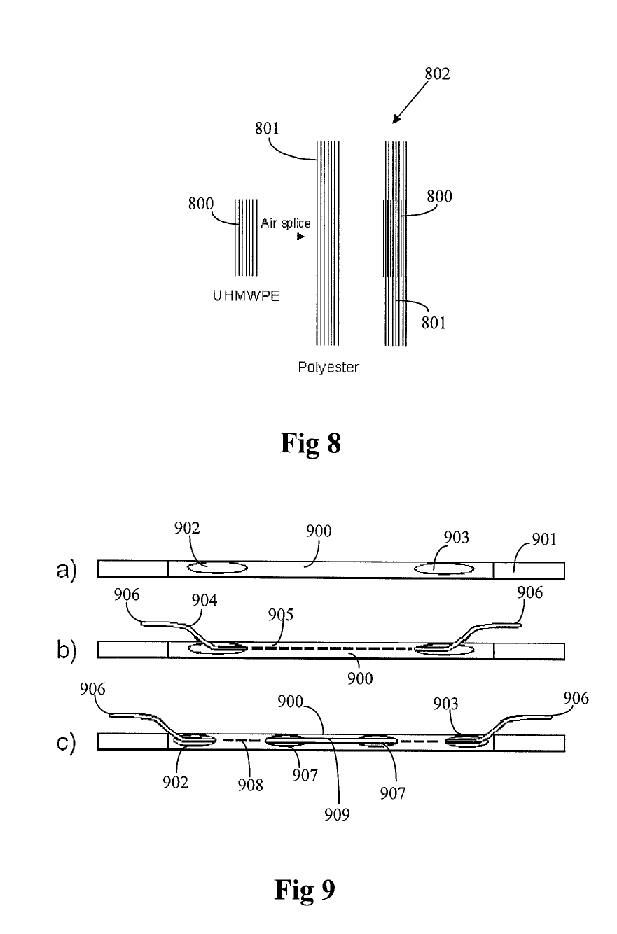

FIG. 8 illustrates a variation on the formation of the medical cord of FIG. 4;

FIG. 9 illustrates schematically the medical cord comprising a biological tissue housed or partly housed within the cord structure;

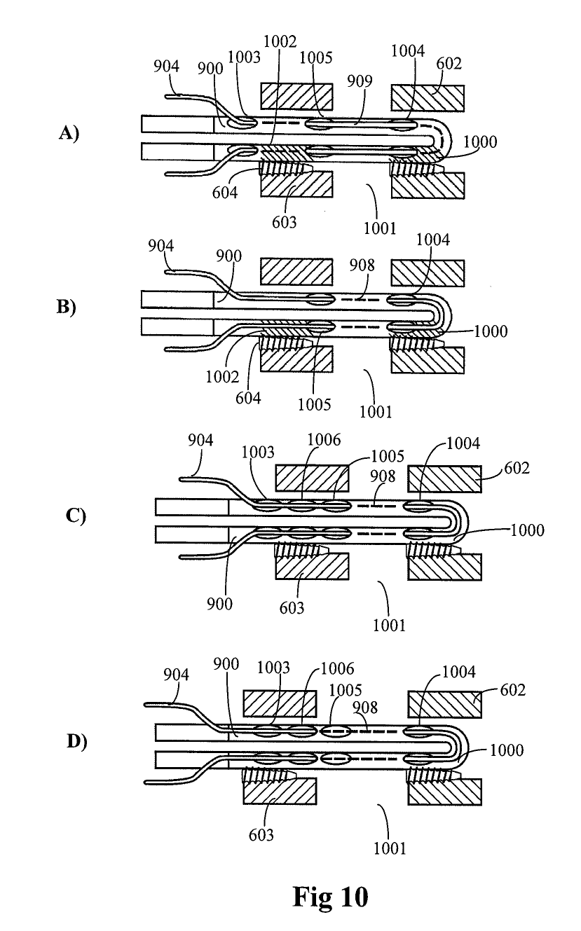

FIG. 10 illustrates the cord and biological tissue core of FIG. 9 anchored in position at a plurality of anchor sites according to four specific implementations of the present invention.

Referring to FIG. 1, the surgical cord 100 comprises a main length 101 bordered at each end by end regions 102. Main length 101 comprises a substantially tubular hollow structure in which overlapping yarn strands form tubular walls 103 to define a central hollow cavity 104. The yarn strands comprise a plurality of filaments twisted together along their length to provide cohesion. According to the specific implementation, the yarn strands are interwoven to form a mesh-like structure comprising warps that extend substantially parallel to the longitudinal axis of the cord and wefts aligned substantially transverse to the cord longitudinal axis. Each end region 102 comprises a flat substantially planar cross section 105. The substantially planar end regions 105 may be formed by flattening end regions 102. Alternatively, end regions 102 are non-tubular 106 and are formed as a substantially planar tape like structure from overlapping yarns.

Referring to FIG. 2, surgical cord 100 comprises regions over its length having different physical and/or mechanical properties. The cord comprises a first material 200 extending over the main length and end regions 102. An intermediate material, coating, manufacturing process, surface treatment or change in structure 201 forms the medical cord along its length between the material of main length 200 and end regions 102 to provide a composite material cord. The cord therefore may be regarded as having segments of different material or physical and/or mechanical properties extending over discrete regions along its length.

According to further specific implementations, material 201 may be overlaid on top of material 200. These discrete regions along the length of the cord provide sections exhibiting different mechanical and/or physical properties such as extensibility, stiffness, and abrasion resistance, for example.

Changes in material along the length of the cord may be provided at any point along the cord length and in particular at the region of the interface 106 between end regions 102 and main length 101.

Referring to FIG. 3, the yarn strands are formed by interconnecting loops 300 to 307. Each loop is formed as a continuous loop during a preassembly process involving winding, twisting and entwining filaments to form the respective loops. The different materials within the cord are incorporated as an integral part of the cord body by interconnecting a loop 300 of first material with a loop 301 of a second material. As illustrated in FIG. 3, the yarns used to construct the cord may be assembled according to a plurality of different interconnecting loop arrangements to provide the required strength, flexibility, extensibility, stiffness and material density.

FIG. 4 illustrates an alternative cord assembly process in which a first material 400 is air spliced with a second material 401 to form the composite medical cord 402. The air splicing process provides an overlap region 403 forming the junction between first material 400 and second material 401.

FIG. 5 illustrates variation on the alignment of the interface between the first material and second material formed by loops 300, 301, respectively. Referring to (b) and (c) interface 500 may be aligned transverse to the longitudinal axis of the cord so as to stagger the interface in the direction of the cord main length. This configuration is advantageous to achieve the desired tensile strength at the interface in response to load. The length of loops 300 may be substantially equal as illustrated in (b) or the respective lengths may be different across the width of the cord so as to provide a tapered region of different material. As will be appreciated, by variation of the respective lengths of loops 300, it is possible to create regions of different material, extending along the length of the cord, having a different shape profiles. Referring to (d) interface 501 may be aligned perpendicular to the longitudinal axis of the cord length. According to one embodiment (a) the cord may be formed by interlinking loops of different cross sectional sizes 300, 301 so as to provide the desired mechanical and/or physical properties at discrete sections of cord along its length.

FIG. 6 is a schematic illustration of the medical cord comprising multiple changes of the filament material in the longitudinal axis. Cord 101 comprises first material 608 and second material 600, 601 formed as three discrete annular bands along the main length of cord 101. According to (b) the cord is then folded back on itself and located between first and second tissue sites 602, 603. By way of example, sites 602, 603 may represent bone sites or alternatively sites 602, 603 may represent soft tissue sites or a combination of bone/soft tissue sites. In this folded configuration, the cord material extending between biological anchor sites 602, 603 (region 605) comprises first material 608 whilst second material 600, 601 is positioned adjacent anchor sites 602, 603. Material 608 is configured to simulate the mechanical/physical properties of the ligament or tendon whilst material 600, 601 may exhibit greater abrasion resistance and stiffness. As illustrated in (d) the abrasion resistance properties are advantageous at the regions adjacent anchor sites 602, 603 where for example anchoring screws 604 are utilised to secure cord 101 in position, such that the adjacent abrasion resistance material 608 prevents the threads on the screws 604 cutting the device. The mechanical and physical properties of cord 101 are therefore optimised to suit its specific application. In particular, inextensible material 600, 601 and/or extensible material 608 may be configured to facilitate bone ingrowth. Folding the tissue back on itself is advantageous as conventional fixation devices may be utilised to secure the cord to the bone anchor. Referring to (e) cord 101 may be secured to the tissue site 603 by looping region 601 about an anchoring pin 609. Referring to (f) the looped end 601 may be secured at a bone site 614 via an additional anchoring loop 611 threaded through a bone tunnel 614 and secured in position via a retaining button 610 located externally to the bone tunnel 613 at an opposite end to looped portion 601. Alternatively, and referring to (g) the looped end 601 may be threaded through an anchoring button 612 that spans the gap between bone anchor sites 602, 603. Alternatively, the chord may be used in a non-folded configuration as illustrated in (h). Alternatively, chord 101 may be secured to the bone anchors 603 by folding cord 101 a plurality of times round suitable anchoring devices which may be pins 609 or buttons 611, 610 or 612. As illustrated in (i) the cord may be folded 615 around first anchoring pin 609, folded 616 around a second anchoring pin 609 and then finally folded 617 around the first anchoring pin 609. As will be appreciated, the cord 101 may be folded a plurality of times around such anchoring pins 609 so as to provide a secure fixation for cord 101 at bone anchor sites 603. As will be appreciated, the ends 102 of the cord 101 may be knotted together over the anchoring device 609 to further enhance fixation. As will be appreciated, the cord can be used with combinations of different anchors at the tissue sites.

Referring to FIG. 7 the medical cord may comprise different materials 700, 701, 702 incorporated within cord to comprise different cross sectional profiles along the cord length. For example, regions 701 may comprise a greater cross sectional profile than a central region and respective end regions. The more bulky intermediate regions 701 may be formed by interlinking loops 703 and 704 where loops 704 comprise more filaments to create the increased cross sectional profile.

As illustrated in (b) the change in cross sectional profile along the length of the cord may be used as a means to anchor the cord in position. For example, when inserted within a bone tunnel comprising a first diameter 706 and a reduced diameter 707, the larger cross sectional profile portion 701 would abut the constriction 708 formed by the change in diameters 706, 707 within bone 705. This would prevent the cord from completely pulling through bone 705.

FIG. 8 illustrates a further example of cord assembly in which a first material 800 is overlaid by air splicing on a second material 801 to form a composite structure 802.

FIG. 9 illustrates a further specific implementation in which the tubular cord comprises a main length 900 and end regions 901. First and second apertures 902, 903 are formed within the tubular walls to provide a means of access to the internal cavity extending longitudinally within main body 900. This tubular cavity is configured to house or at least partially house an additional separate core material 904. Material 904 may comprise biological tissue including ligaments and tendons. According to the example (b), tissue 904 is threaded through apertures 902, 903 such that end portions 906 of tissue 904 are positioned external to the tubular body 900 whilst a central portion 905 of tissue 904 extends within tubular body 900. Referring to (c), the cord may comprise a plurality of apertures 902, 907, 903 extending along its main length. Tissue 904 may be introduced within the tubular body 900 via apertures 902, 907, 903 such that discrete regions 909 of tissue 904 may extend externally along the cord length whilst regions 908 may extend internally within the tubular body 900.

FIG. 10 illustrates the medical cord of FIG. 9 positioned between first and second anchor sites 602, 603 as detailed with reference to FIG. 6. The tubular cord provides a protective sheath for the biological tissue core. This is particularly advantageous to protect tissue 904 at regions 1000 and 1002 from the sharp edges of anchoring screws 604 and the frictional contact with bone anchor sites 602, 603. According to the embodiment (a) of FIG. 10, tissue 909 is exposed at region 1001 (between anchor sites 602, 603) being external to the protective sheath 900. Alternatively, according to the embodiment (b) of FIG. 10, tissue 908 is sheathed at region 1001 (between anchor sites 602, 603) being housed within the protective sheath 900. The material of the prosthetic cord may also be optimized at these anchor site regions 602, 603 to provide the desired abrasion resistance, stiffness etc.

Referring to the embodiments of FIG. 10(a) biological tissue 904 is threaded into the hollow sheath 900 through apertures 1003 to emerge at apertures 1005. The biological core then re-enters sheath 900 via apertures 1004. In the alternative embodiment referring to FIG. 10(b) the sheath 900 is devoid of intermediate apertures 1005 such that the cord is threaded through apertures 1005 to re-emerge from apertures 1004.

According to the further embodiments of FIG. 10(c) and (d) a fourth aperture 1006 is provided. This configuration allows the surgeon greater flexibility when implanting the prosthetic within patients of different sizes. For example, for a smaller patient illustrated schematically in FIG. 10(c) the biological core 904 exits the prosthetic at aperture 1005 as the distance 1001 between the bone sites 602 and 603 is relatively small. For a larger patient illustrated schematically in FIG. 10(d) the biological core would not exit the sheath until aperture 1006 (or even 1003), so as to be protected throughout the full length of region 1001 between the bone sites by the sheath 900. Accordingly, the present implantable prosthetic may comprise a plurality of apertures extending along the length of the hollow sheath through which a biological tissue core may be threaded selectively. The present prosthetic is therefore configured to suit a variety of different applications and to extend between bone anchorage sites separated by a variety of different distances. A further specific implementation to that of FIG. 10 comprises an elongate opening to replace the plurality of apertures 1003 to 1006. With this embodiment, the elongate opening could be suture closed by a surgeon after the ligament or tendon 904 has been threaded and pulled through the sheath 900 over the appropriate distance.

The examples referred to in FIGS. 1 to 10 describe, in part, a change in material along the length of the cord to provide different regions exhibiting different mechanical and/or physical properties. Further embodiments, to optimise the mechanical and/or physical properties at discrete regions along the length of the cord may involve altering the pattern, density, thickness and shape of the cord and/or the component yarn strands and filaments, applying a coating, manufacturing process or surface treatment. If different material is incorporated within the cord along its length, this different material may replace sections of material or may be integrated into the material structure by conventional textile techniques including weaving, braiding, plying, knitting etc.

1. A surgical cord comprising:
a plurality of overlapping yarn strands forming the walls of a substantially tubular region extending over a length of the cord.

2. The cord as claimed in claim 1 wherein said overlapping yarn strands are woven comprising a plurality of warps extending substantially parallel to the longitudinal axis of the cord and wefts aligned transverse to said longitudinal axis.

3. The cord as claimed in claim 1 wherein said yarn strands comprise a plurality of different materials to create sections of different material along the length of said cord.

4. The cord as claimed in claim 3 wherein said cord comprises annular bands of the different material extending over sections of said tubular region.

5-7. (canceled)

8. The cord as claimed in claim 3 wherein said yarn strands comprise a first material being a synthetic polymer and a second material being bioabsorbable.

9. The cord as claimed in claim 8 comprising a biologically active substance.

10. The cord as claimed in claim 3 wherein said yarn strands comprise a first material and a second material wherein said first material is more extensible than said second material.

11. The cord as claimed in claim 3 wherein said yarn strands comprise a first material and a second material wherein said first material comprises a greater abrasion resistance than said second material.

12. The cord as claimed in claim 3 wherein said yarn strands comprise a first material and a second material wherein said first material comprises a stiffness greater than said second material.

13. The cord as claimed in claim 3 wherein said sections are separate along the length of said cord.

14-15. (canceled)

16. The cord as claimed in claim 2 wherein a density of said weave is different at different regions along the length of said cord, the density difference being due to a change in the spacing between a woven structure created by the woven warps and wefts.

17. The cord as claimed in claim 1 wherein said yarn strands comprise different thicknesses.

18. The cord as claimed in claim 1 wherein a repeating pattern by which said yarn strands are overlapped is different at different regions along the length of said cord.

19. The cord as claimed in claim 2 comprising a different number of warps at different regions along the length of said cord.

20. The cord as claimed in claim 2 comprising a different number of wefts at different regions along the length of said cord.

21. The cord as claimed in claim 1 wherein said yarn strands comprise a coating at different regions along the length of the cord.

22. The cord as claim in claim 1 wherein the thickness and/or cross sectional profile of the yarn strands is different at different regions along the length of the cord.

23. The cord as claimed in claim 1 wherein said yarn strands comprise different physical/mechanical properties at different regions along the length of the cord.

24. The cord as claimed in claim 2 wherein said yarn strands are formed as loops and said warps are formed by interlinking said loops.

25-27. (canceled)

28. The cord as claimed in claim 1 comprising at least one aperture formed within the walls of said tubular region.

29. The cord as claimed in claim 28 comprising biological tissue positioned within said tubular region, said tissue being substantially elongate and extending through said at least one aperture.

30. The cord as claimed in claim 29 comprising a plurality of apertures, said biological tissue extending through at least some of said apertures along the length of said tubular region such that the biological tissue is housed within separate sections along the length of said tubular region.

31-46. (canceled)

47. The cord as claimed in claim 1 wherein said tubular region is boarded at each end by a substantially flat region formed by said overlapping yarn strands, said flat region being substantially planar relative to said tubular region.
